(19) United States
(12) Patent Application Publication
(10) Pub. No.: US 2018/0074034 A1
(43) Pub. Date: Mar. 15, 2018

(54) Vehicle Identification System and Associated Methods

(71) Applicant: Wal-Mart Stores, Inc., Bentonville, AR (US)

(72) Inventors: Nicholaus Adam Jones, Fayetteville, AR (US); Robert James Taylor, Rogers, AR (US); Aaron James Vasgaard, Fayetteville, AR (US); Matthew Allen Jones, Bentonville, AR (US)

(21) Appl. No.: 15/700,387

(22) Filed: Sep. 11, 2017

Related U.S. Application Data
(60) Provisional application No. 62/393,225, filed on Sep. 12, 2016.

(51) Int. Cl. G01N 33/00; G06Q 30/02; G07C 5/00

(52) U.S. Cl. CPC G01N 33/0075; G07C 5/008; G06Q 30/02

(57) ABSTRACT

A vehicle identification system that includes exhaust emissions sensors that identify a vehicle type based on detected exhaust emission profiles and further determines vehicle stay duration and related metrics is discussed. Additional audio sensors may be used to further identify a change in load on the vehicle due to purchases made at the retail facility. The exhaust emission profiles detected by the vehicle identification system can be correlated with transaction data for the customer to determine the exact items purchased by the customer.

### CROSS-REFERENCE TO RELATED APPLICATIONS

This application claims the benefit of co-pending, commonly assigned U.S. Provisional Patent Application No. 62/393,225, which was filed on Sep. 12, 2016. The entire content of the foregoing provisional patent application is incorporated herein by reference.

### BACKGROUND

A large number of customers visit retail establishments each day. Each customer purchases different products and different amounts of products. In addition, each customer visits the retail establishment at different times of the week or month. The more information a retail establishment can gather regarding the customers visiting the retail establishment, the better the retail establishment can accommodate the customers.

### SUMMARY

Exemplary embodiments of the present invention provide a vehicle identification system that determines a vehicle type by detecting exhaust emissions of a vehicle visiting a location of interest. The vehicle identification system may determine the duration and related metrics of visits of a customer to an establishment and correlates those visits to transaction data to accumulate customer demographic data for the establishment's benefit. Additional audio and other sensors may be used to further identify a change in load on the vehicle due to purchases made at the establishment.

In one embodiment, an exemplary vehicle identification system includes one or more exhaust emissions sensors and an emissions analysis system. The one or more exhaust emissions sensors are positioned to detect a first exhaust emission of a vehicle. The emissions analysis system is in electronic communication with and configured to receive data associated with the detected first exhaust emission from the one or more exhaust emissions sensors. The emissions analysis system includes a location database, an exhaust profile database, a processor, and memory. The location database stores a geographic location of each of the one or more exhaust emissions sensors. The exhaust profile database stores known exhaust emission profiles with each of the plurality of known exhaust emission profiles being associated with a known vehicle type. The memory includes instructions for an emissions analysis module that, when executed by the processor, causes the emissions analysis system to determine a first detected exhaust emission profile from the data associated with the detected first exhaust emission. The memory further includes instructions for an emissions analysis module that, when executed by the processor, causes the emissions analysis system to identify one of the known exhaust emission profiles stored in the exhaust profile database as a corresponding profile to the first detected exhaust emission profile. The memory also includes instructions for an emissions analysis module that, when executed by the processor, causes the emissions analysis system to determine a vehicle type associated with the corresponding profile as the vehicle type for the vehicle. The memory further includes instructions for an emissions analysis module that, when executed by the processor, cause the emissions analysis system to add the vehicle type to a stored set of data associated with a location at which the first exhaust emission was detected.

In another embodiment, an exemplary method for vehicle identification includes detecting, at one or more exhaust emissions sensors, a first exhaust emission of a vehicle. The method also includes receiving, at an emissions analysis system in electronic communication with the one or more exhaust emissions sensors, data associated with the detected first exhaust emission. The method includes determining, via an emissions analysis module of the emissions analysis system, a first detected exhaust emission profile from the data associated with the detected first exhaust emission. The method additionally includes identifying one of the known exhaust emission profiles stored in an exhaust profile database as a corresponding profile to the first detected exhaust emission profile. Additionally, the method includes determining a vehicle type associated with the corresponding profile as the vehicle type of the vehicle and adding the vehicle type to a stored set of data associated with a location at which the first exhaust emission was detected.

In an embodiment, an exemplary non-transitory medium storing computer-executable instructions for vehicle identification is provided. The instructions, when executed, cause at least one processing device to detect, at one or more exhaust emissions sensors, a first exhaust emission of a vehicle. The instructions, when executed, also cause the at least one processing device to receive, at an emissions analysis system in electronic communication with the one or more exhaust emissions sensors, data associated with the detected first exhaust emission and to determine, via an emissions analysis module of the emissions analysis system, a first detected exhaust emission profile from the data associated with the detected first exhaust emission. The instructions, when executed, also cause the at least one processing device to identify one of the known exhaust emission profiles stored in an exhaust profile database as a corresponding profile to the first detected exhaust emission profile and to determine a vehicle type associated with the corresponding profile as the vehicle type of the vehicle. The instructions, when executed, further cause the at least one processing device to add the vehicle type to a stored set of data associated with a location at which the first exhaust emission was detected.

It should be appreciated that combinations and/or permutations of embodiments are envisioned as being within the scope of the present invention. Other objects and features will become apparent from the following detailed description considered in conjunction with the accompanying drawings. It is to be understood, however, that the drawings are designed as an illustration only and not as a definition of the limits of the present disclosure.

### BRIEF DESCRIPTION OF THE DRAWINGS

To assist those of skill in the art in making and using the disclosed vehicle identification systems and associated methods, reference is made to the accompanying figures. The accompanying figures, which are incorporated in and constitute a part of this specification, illustrate one or more embodiments of the invention and, together with the description, help to explain the invention. In the figures:

FIG. 1 is a block diagram of an exemplary vehicle identification system in an embodiment.

FIG. 2 is a block diagram of an exemplary emissions database of a vehicle identification system in an embodiment.

FIG. 3 is a block diagram of an exemplary engine sound database of a vehicle identification system in an embodiment.

FIG. 4 is a block diagram of an exemplary sensor environment of a vehicle identification system in an embodiment.

FIG. 5 is a block diagram of an exemplary database system of a vehicle identification system in an embodiment.

FIG. 6 is a block diagram of a computing device in accordance with exemplary embodiments.

FIG. 7 is a block diagram of an exemplary vehicle identification system environment in accordance with an embodiment.

FIG. 8 is a flowchart illustrating an implementation of an exemplary vehicle identification system in accordance with an embodiment.

FIG. 9 is a flowchart illustrating an implementation of an exemplary vehicle identification system in accordance with an embodiment that identifies a shopping time for the vehicle.

FIG. 10 is a flowchart illustrating an implementation of an exemplary vehicle identification system in accordance with an embodiment that identifies a path of the vehicle.

FIG. 11 is a flowchart illustrating an implementation of an exemplary vehicle identification system in accordance with an embodiment utilizing audio sensors.

### DETAILED DESCRIPTION

It should be understood that the relative terminology used herein, such as “front”, “rear”, “left”, “top”, “bottom”, “vertical”, “horizontal”, “up” and “down” is solely for the purposes of clarity and designation and is not intended to limit embodiments to a particular position and/or orientation. Accordingly, such relative terminology should not be construed to limit the scope of the present disclosure. In addition, it should be understood that the scope of the present disclosure is not limited to embodiments having specific dimensions. Thus, any dimensions provided herein are merely for an exemplary purpose and are not intended to limit the invention to embodiments having particular dimensions.

Exemplary embodiments of the present invention provide a vehicle identification system that identifies vehicles of individuals making purchases visiting an establishment by detecting the exhaust emissions of the vehicle. In particular, determining the duration and/or frequency of visits of the customer to the retail establishment and the amount of purchases made by the customer can be helpful in determining the types of customers visiting the retail establishment. The exemplary vehicle identification system includes exhaust emissions sensors that identify the vehicle type based on the detected exhaust emission profile, and, when combined with audio sensors, can further determine a change in load on the vehicle due to purchases made at the establishment based on a change in detected engine sounds. The exhaust emission profiles detected by the vehicle identification system can also be correlated with transaction data for the customer to determine the exact items purchased by the customer operating the vehicle.

Although discussed herein as a system used at an establishment where individuals make purchases, it should be understood that the exemplary system can be used in a variety of applications. As one example, the exemplary system can be used as a security measure to determine improper border crossing involving individuals and/or items. For example, the system can determine excessive loads or changes in load on vehicles crossing the border based on the detected emissions and/or audio to determine whether suspicious activities are taking place such as undeclared persons or items being in the vehicle.

The emissions analysis system of the exemplary vehicle identification system can track the vehicles based on the time of entry and exit from a predetermined geographic area, and based on the detected emissions can classify the vehicle based on size, e.g., small/compact, sport utility vehicle (SUV), pickup truck, minivan, or the like. The emissions analysis system can determine the length of time spent at the retail establishment, the type of vehicle, and can correlate transaction data to determine which purchases were made by customers in specific vehicles. An engine sound analysis system includes audio sensors to determine revolutions per minute (RPMs) of the vehicle engine and can be used in conjunction with the emission sensors to determine a change in load of the vehicle resulting from customer purchases.

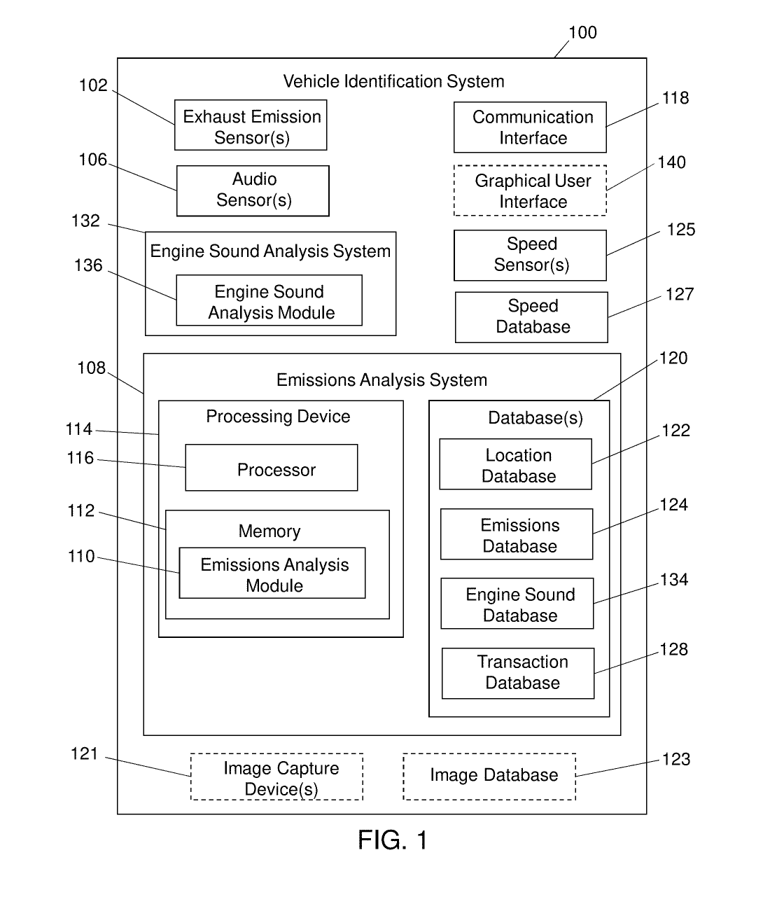

FIG. 1 is a block diagram of an exemplary vehicle identification system 100 (hereinafter “system 100”) in accordance with exemplary embodiments. The system 100 generally includes one or more exhaust emission sensors 102 disposed within a predetermined geographic area (e.g., property surrounding an establishment, such as the parking lot of a retail establishment in which customers can park their vehicles). Each of the exhaust emission sensors 102 may be configured to detect an initial exhaust emission (i.e., a first exhaust emission) and later a subsequent exhaust emission (i.e., a second exhaust emission). For example, each of the exhaust emission sensors 102 can be configured to detect an exhaust emission of vehicles entering an area near the establishment and an exhaust emission of vehicles exiting the area.

The system 100 may further include one or more audio sensors 106 disposed within the area near the establishment. Each of the audio sensors 106 can be configured to detect first engine sounds and subsequently second engine sounds indicative of the sound of the engine's RPMs. For example, the engine sound of vehicles entering the area in the vicinity of the establishment and the engine sounds of vehicles exiting the area may be determined and compared to determine a change between the sounds that indicates the vehicle is working harder due to a greater load.

The system 100 includes an emissions analysis system 108 and a processing device 114 equipped with a processor 116. The emissions analysis system 108 includes a memory 112 and an emissions analysis module 110. The emissions analysis module 110, can be executed on a processing device 114 such as a computing device or other electronic device. In an embodiment, the emissions analysis module 110, can also be executed on a different processing device including a processor. The emissions analysis system 108 is in electronic communication with the exhaust emission sensors 102, and is configured to receive data associated with the detected exhaust emissions from the exhaust emission sensors 102 via, e.g., a communication interface 118, through wired and/or wireless channels.

The system 100 generally includes one or more databases 120. The database 120 is in electronic communication with the emissions analysis system 108. The database 120 can include a location database 122. Although illustrated as component of the emissions analysis system 108, in an embodiment, the location database 122 can be separate from the emissions analysis system 108. The location database 122 electronically stores information corresponding to the exhaust emission sensors 102 and the audio sensors 106 within the geographic area. For example, the location database 122 can include information relating to the type of sensor, the operation status of the sensor, and/or the geographic location of the sensor within the geographic area in the vicinity of an establishment of interest.

The database 120 includes an emissions database 124. The emissions database 124 includes a plurality of known exhaust emission profiles for vehicles. Each of the plurality of known exhaust emission profiles can be associated with a known vehicle type (e.g., small/compact, sport utility vehicle (SUV), pickup truck, minivan, or the like). The memory 112 of the emissions analysis system 108 includes instructions for the emissions analysis module 110 that can be executed by the processing device 114. The emissions analysis system 108 determines a detected exhaust emission profile from the data associated with the detected exhaust emission of the vehicle. The emission analysis system 108 further identifies one of the known exhaust emission profiles stored in the emissions database 124 as a corresponding profile to the detected exhaust emission profile. The emissions analysis system 108 determines a vehicle type associated with the identified corresponding profile as the vehicle type of the vehicle for which the exhaust emission was detected. For example, based on a detected exhaust emission for a vehicle entering the area near an establishment and the known exhaust emission profiles stored in the emissions database 124, the emissions analysis system 108 can determine the type of vehicle being driven by a customer. The emissions analysis system 108 further adds the vehicle type to a stored set of data associated with a location at which the exhaust emission was detected for the vehicle.

Different types of vehicles generally have different exhaust emissions profiles that are electronically stored in the emissions database 124. In an embodiment, the profiles stored in the emissions database 124 can be in the form of ranges of normal emissions for different types of cars. For example, cars having a carburetor and cars having a fuel injection engine generally have different emissions profiles. In an embodiment, the exhaust emission sensors 102 can be, e.g., optical sensors, mass spectrometers, combinations thereof, or the like. In an embodiment, the exhaust emission sensors 102 can detect particulates, e.g., carbon monoxide, carbon dioxide, ammonia, water vapor, nitrogen oxides, other particulate matter, combinations thereof, or the like, and the amount of such particulates in the detected exhaust. The particulate profile for each vehicle can therefore be determined by the system 100 during entry of the vehicle and before purchased items are placed in the vehicle.

In an embodiment, the system 100 can include one or more image capture devices 121 (e.g., video camera, still image camera, or the like) configured to capture one or more still images and/or videos of the vehicle entering the location in which the exhaust emission is being detected. The still images and/or videos captured by the image capture devices 121 can be stored in an image database 123. In an embodiment, the image capture devices 121 can be used to confirm the accuracy of the type of vehicle determined by the emissions analysis system 108 based on the exhaust emission profile matching process.

Subsequently, the exhaust emission sensor 102 detects a second exhaust emission of the vehicle. For example, a second exhaust emission may be detected by the exhaust emission sensors 102 after a customer's completion of shopping at a retail establishment (e.g., when the customer is leaving the retail establishment). The emissions analysis system 108 determines a second detected exhaust emission profile from data associated with the detected exhaust emission. The emissions analysis system 108 may then identify the detected exhaust emission as belonging to a specific (earlier identified) vehicle based on identifying the previously determined corresponding profile as also corresponding to the second detected exhaust emission profile. In an embodiment, the image capture devices 121 can be used to confirm that the second exhaust emission detected by the exhaust emission sensor 102 is associated or correlated with the same vehicle as data representative of the detected first exhaust emission during entry of the vehicle to the retail establishment.

In some embodiments, changes in emission characteristics between the first and second detected emissions may provide information on vehicle owner activities. For example, in one embodiment, during exit of the vehicle from an area near an establishment of interest, the exhaust emissions sensor 102 may detect an exhaust emission of a vehicle and the vehicle identification system may match it to an earlier detected vehicle whether through similar emission characteristics or video analytics (e.g., using the image capture devices 121). The system may also identify a change between the detected first and second exhaust emission for the identified vehicle indicative of an increased load on the vehicle as a result of the vehicle storing items that were purchased at the retail establishment during the visit by the customer (e.g., when the added weight within the vehicle results in an increase in the exhaust emission).

As an example, during entry and prior to purchase of items, a vehicle with a 2.0 L engine at approximately 500 engine RPMs is expected to have a specific amount of emissions particulates (e.g., a range of particulates). Such emissions particulate profile can be detected and associated with the vehicle upon entry to the retail establishment. When purchased items are placed in the vehicle, the increased weight in the vehicle increases the load on the vehicle, resulting in higher engine RPMs. For example, the vehicle with the 2.0 L engine may travel at approximately 600 engine RPMs after purchases have been made and loaded into the vehicle. The increased engine RPMs result in a greater particulate count detected by the exhaust emissions sensor 102. The difference in the detected emissions particulate profile of the same vehicle before and after loading with purchased items can be correlated to the weight of items purchased. In an embodiment, historical correlated data can be used in a machine-learning manner to estimate the weight of items purchased by customers.

In an embodiment, the database 120 may include a transaction database 128. The transaction database 128 can include information corresponding to transactions at a computational device, such as a point-of-sale terminal including a cash drawer and transaction receipt roll at a retail establishment of interest, including customer names, items purchased, time of purchase, or the like. In an embodiment, the emissions analysis system 108 can electronically retrieve (e.g., through the communication interface 118) from the transaction database 128 transaction data associated with a purchase of products. The transaction data may be for a purchase completed subsequent to the detection of the first exhaust emission of the vehicle detected by the exhaust emission sensors 102 and prior to detection of the second exhaust emission of the vehicle. The vehicle identification system 100 can associate the transaction data with the stored data for a specific vehicle. For example, a correlation engine executed by the processing device 114 can correlate transaction data with the detected emissions of a vehicle, such that a correlation can be determined between the amount of products purchased by a customer in the establishment and an identified vehicle. The detected exhaust emissions, determined vehicle type, determination of whether products were purchased at the establishment, and/or correlation of transaction data can be displayed to a user of the system 100 (e.g., a manager or associate of the retail establishment) via a graphical user interface (GUI) 140.

In an embodiment, the system 100 can include an engine sound analysis system 132. In an embodiment, the engine sound analysis system 132, including the engine sound analysis module 136, can be executed on the processing device 114. In an embodiment, the engine sound analysis system 132, including the engine sound analysis module 136, can also be executed on a different processing device including a processor instead of or in addition to being executed on processing device 114. In an embodiment, the engine sound analysis module 136 can be executed by an identification engine 126 executing on the processing device 114. The engine sound analysis system 132 is in electronic communication with the audio sensors 106, and can receive data associated with the detected engine sounds from the audio sensors 106. For example, the audio sensors 106 can detect engine sounds from vehicles in a geographic area near an establishment of interest, and the detected engine sounds can be electronically transmitted to the engine sound database 134 of database 120. The detected engine sounds can further be analyzed by the engine sound analysis module 136.

The engine sound database 134 can include known engine sound profiles. Each of the known engine sound profiles can be associated with a known vehicle type (e.g., small/compact, sport utility vehicle (SUV), pickup truck, minivan, or the like). Based on the engine sound data from the detected engine sound of the vehicle (e.g., a first engine sound upon entry into the geographic area), the engine sound analysis module 136 determines a first detected engine sound profile of the vehicle. For example, the first detected engine sound profile can correspond with the RPMs of the engine before the customer has made purchases at the retail establishment. Based on a second sound data from a detected second engine sound of the vehicle, the engine sound analysis module 136 may determine a second detected engine sound profile. For example, the second detected engine sound profile can correspond with the RPMs of the engine of the vehicle after the customer has made purchases at the retail establishment and is exiting the geographic area. The detected RPMs can be correlated with the detected speed of the vehicle as measured by the speed sensors 125, such that the first and second engine sound profiles are detected at substantially similar RPMs. By detecting the RPMs of the vehicle traveling at the same speed, a change in the RPMs can be directly correlated with a change in weight of the contents of the vehicle (e.g., whether due to additional passengers and/or purchased products).

The engine sound analysis module 136 may identify a corresponding engine sound profile in the engine sound profile database of the engine sound database 134 as a corresponding profile to the first and second detected engine sound profiles of the vehicle in order to identify the type of vehicle (e.g., small/compact, sport utility vehicle (SUV), pickup truck, minivan, or the like) based on the first and second detected engine sound profiles. The engine sound analysis module 136 identifies a change in engine RPMs based on a change between the first and second detected engine sound profiles, and further determines a change in weight of the vehicle based on that change in engine RPMs. The change in weight can indicate that the customer made purchases at the retail establishment.

In an embodiment, the system 100 can include one or more speed sensors 125 configured to detect the speed of each vehicle at entry and exit points of the area associated with the retail establishment. The detected speed can be electronically stored in a speed database 127. The speed sensors 125 can operate in combination with the audio sensors 106 to identify the engine RPMs of each vehicle at specific speed(s) during entry, and the engine RPMs of each vehicle at the same speed(s) during exit from the retail establishment. If an individual did not make purchases at the retail establishment, the engine RPMs at entry and exit for the same speed of the vehicle should be approximately equal. However, if purchases were made at the retail establishment, the engine RPMs at exit would be detected to be higher than the engine RPMs at entry if the vehicle is traveling at the same speed. Such change in engine RPMs can be used to estimate the change in weight due to purchases at the retail establishment.

For example, a first detected engine sound profile of a vehicle can be a low engine RPM level, while a second detected engine sound profile of the vehicle can be a higher engine RPM level (e.g., at the same travel speed). The higher engine RPMs, caused by the engine working harder, may indicate a higher weight or load within the vehicle, further indicating that the customer made purchases at the retail establishment and the higher weight or load is caused by the products placed within the vehicle. In an embodiment, a correlation engine can correlate the change in engine RPMs with the transaction data from the transaction database 128 to determine the products purchased by the owner of the vehicle for which the engine RPMs were measured. The detected change in engine RPMs can thereby be correlated with a specific amount and weight of products purchased by the customer. The detected engine sound profiles, change in engine RPMs, determined vehicle type, and/or the indication of a weight or load change between the detected engine sound profiles can be displayed to a user of the system 100 via the GUI 140.

The exemplary vehicle identification system 100 can thus be used to obtain demographic information regarding customers visiting the establishment without directly involving customers in the process. In some embodiments, the vehicle identification system 100 can determine who is shopping in the establishment, the number of family members and or customers in the vehicle, the residential location of the customers relative to the establishment, the type of vehicle driven by the family, the amount of items purchased at the retail establishment, combinations thereof, or the like. It will be appreciated that not all of these types of information are gleaned solely from emission sensor readings but rather, for some information, may be determined by the vehicle identification system 100 using the emission sensor reading in combination with other available information associated with the customers including information gained through the use of additional types of sensors such as the audio sensors described above.

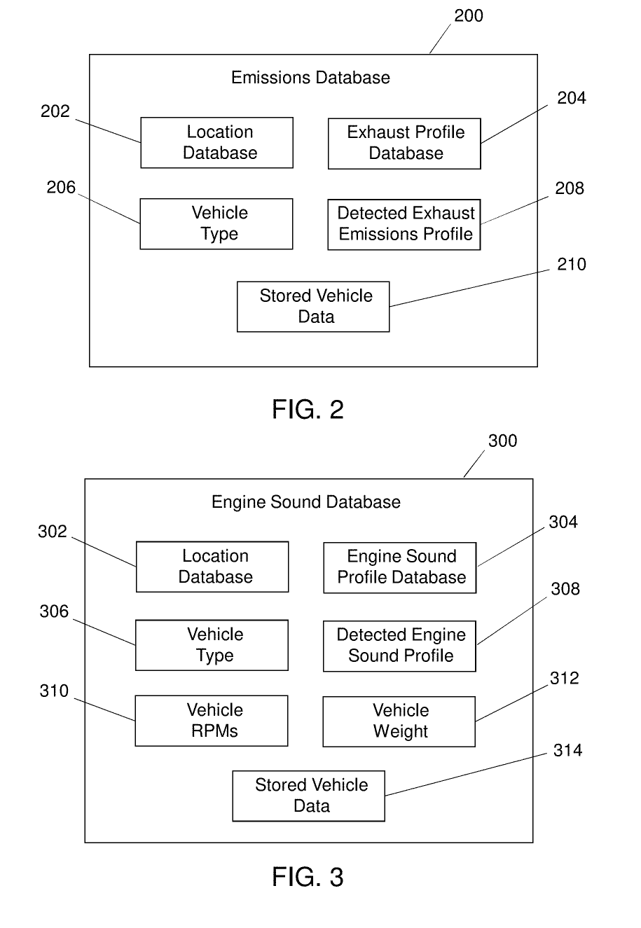

FIG. 2 is a block diagram of an exemplary emissions database 200 (e.g., the emissions database 124 of the database 120 of FIG. 1) in an embodiment. The exemplary emissions database 200 includes a location database 202, an exhaust profile database 204, a vehicle type 206, a detected exhaust emission profile 208, and stored vehicle data 210. The location database 202 includes data corresponding to the location of the exhaust emission sensors 102 within a defined geographic area, as well as additional information on each of the exhaust emission sensors 102, such as the sensor name, sensor type, sensor manufacturer, sensor range, or the like. The exhaust profile database 204 includes data regarding known exhaust emission profiles for a variety of vehicles. For example, the exhaust profile database 204 can include exhaust emissions ranges for each type of vehicle (e.g., small/compact, sport utility vehicle (SUV), pickup truck, minivan, or the like) under normal driving conditions and based on the age of the vehicle.

The vehicle type 206 includes data relating to the different types of vehicles for the exhaust profiles stored within the exhaust profile database 204. The detected exhaust emissions profile 208 includes data corresponding to the detected exhaust emissions received from the exhaust emission sensors 102. For example, the detected exhaust emissions profile 208 can include the first detected exhaust emissions profile and the second detected exhaust emissions profile determined based on the detected first and second exhaust emissions of a vehicle. The stored vehicle data 210 can include a compartmentalized storage of information for each of the vehicles for which the exhaust emissions were detected and analyzed, such as the exhaust emissions values, the change in exhaust emissions, the determined vehicle type, the estimated change in weight of the vehicle after purchases were made at the retail establishment, the correlated transaction data, or the like.

FIG. 3 is a block diagram of an exemplary engine sound database 300 (e.g., the engine sound database 134 of the database 120 of FIG. 1) in an embodiment. The exemplary engine sound database 300 includes a location database 302, an engine sound profile database 304, a vehicle type 306, a detected engine sound profile 308, vehicle RPMs 310, vehicle weight 312, and stored vehicle data 314. The location database 302 includes the location of the audio sensors 106 within a defined geographic area near an establishment of interest, as well as additional information on each of the audio sensors 106, such as the sensor name, sensor type, sensor manufacturer, sensor range, or the like. The engine sound profile database 304 includes data regarding known engine sound profiles for a variety of vehicles. For example, the engine sound profile database 304 can include engine sound ranges for each type of vehicle (e.g., small/compact, sport utility vehicle (SUV), pickup truck, minivan, or the like) under normal driving conditions (e.g., for different travel speeds or travel speed ranges) and based on the age of the vehicle.

The vehicle type 306 includes data relating to the different types of vehicles for the engine sound profiles stored within the engine sound profile database 304. The detected engine sound profile 308 includes data corresponding to the detected engine sounds received from the audio sensors 106. For example, the detected engine sound profile 308 can include the first detected engine sound profile and the second detected engine sound profile determined based on the detected first and second engine sounds of a vehicle. The vehicle RPMs 310 can store data corresponding to the detected engine RPMs for different types of vehicles within the defined geographic area and the change in engine RPMs before and after purchases have been made at the establishment. The vehicle weight 312 can store data corresponding to the estimated change in weight of the vehicle based on the change in detected engine RPMs. The stored vehicle data 314 can include a compartmentalized storage of information for each of the vehicles for which the engine sounds were detected and analyzed, such as the engine RPMs, the change in engine RPMs, the determined vehicle type, the estimated change in weight of the vehicle after purchases were made at the establishment, the correlated transaction data, or the like.

In an embodiment, the emissions analysis system 108 and/or the engine sound analysis system 132 can identify a time of detection of the first exhaust emission and/or the first engine sounds as a time of arrival of the vehicle. The emissions analysis system 108 and/or the engine sound analysis system 132 can further identify a time of detection of the second exhaust emission and/or the second engine sounds as a time of departure of the vehicle from the geographic area. The emissions analysis system 108 and/or the engine sound analysis system 132 can further identify a difference between the time of arrival and a time of completion of the purchase as a total shopping time associated with the vehicle. The emissions analysis system 108 and/or the engine sound analysis system 132 can add the total shopping time to the stored vehicle data 210, 314 in the emissions database 200 and/or the engine sound database 300. In an embodiment, the emissions analysis system 108 and/or the engine sound analysis system 132 can determine a total on-site time of the vehicle based on a difference between the time of arrival and the time of departure, and store the total on-site time to the stored vehicle data 210, 314 in the emissions database 200 and/or the engine sound database 300.

In an embodiment, the emissions analysis system 108 and/or the engine sound analysis system 132 can assign the transaction data from the transaction database 128 to a demographic associated with the determined vehicle type. In an embodiment, the emissions analysis system 108 and/or the engine sound analysis system 132 can retrieve from the location database 202, 302 a geographic location of each of the exhaust emission sensors 102 detecting the exhaust emission and/or the audio sensors 106 detecting the engine sounds within the geographic area. The emissions analysis system 108 and/or the engine sound analysis system 132 further identify an instantaneous geographic location of the vehicle based on the geographic locations of the exhaust emission sensors 102 and/or the audio sensors 106. In an embodiment, the emissions analysis system 108 and/or the engine sound analysis system 132 can identify a sequence of instantaneous geographic locations of the vehicle and, based on such information, determine a path transited by the vehicle within the geographic area defined by the exhaust emission sensors 102 and/or the audio sensors 106.

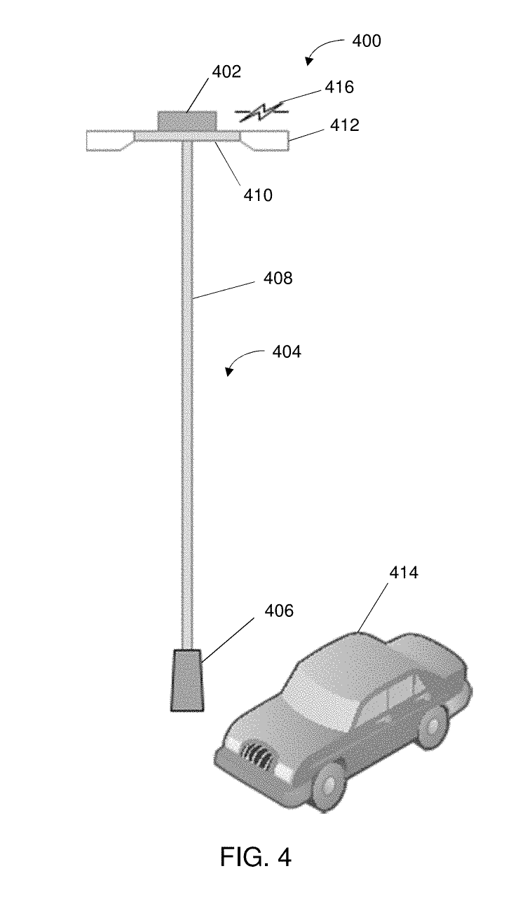

FIG. 4 is a block diagram of an exemplary sensor environment 400 of the system 100. The sensor environment 400 can be disposed within a predetermined geographic area of the system 100. The exhaust emission sensors 102 and/or the sound sensors 106 can be disposed in places within the sensor environment 400 that will maximize the measurement and accuracy of the exhaust emissions and the engine sounds. For example, the exhaust emission sensors 102 and/or the sound sensors 106 can be disposed in parking locations where exhaust is emitted or engine sounds are capable of being detected, along driving lanes within the geographic area, at a vehicle maintenance center (e.g., a tire and lube center), at a garden center loading area, under awnings, at a pharmacy drive through, within or on lights in the parking area, at dedicated monitoring stations, combinations thereof, or the like.

As noted above, the detected data can be used to determine the type of vehicles transiting to the retail establishment, the approximate age of the vehicle, and the load on the vehicle (both by passengers and purchased products). In an embodiment, the audio sensors 106 and/or the exhaust emission sensors 102 can be used to determine the demographics and affluence of the customer population transiting to the retail establishment, and can correlate such data with the customers transiting to the retail establishment versus the population in the area surrounding the retail establishment. For example, as vehicles age, the emissions of the vehicle and/or engine sounds change. Demographics can be estimated by determining the number of older vehicles in the area, and the determination of the type of vehicle in the area can further be used to estimate the types of customers in the surrounding population. For example, a determination that more pickup trucks are transiting to the retail establishment can be used to estimate that the surrounding population may have a large number of construction workers. Such data can be used to generate targeted marketing efforts to address customer gaps in the population.

FIG. 4 illustrates one potential location of a sensor 402 on top of a light assembly 404. However, it should be understood that similar sensors 402 can be disposed in a variety of different locations within the geographic area. The light assembly 404 can include a base 406, a vertical pole 408, a top support beam 410, and one or more lights 412 secured to the top support beam 410. In an embodiment, one or more sensors 402 can be mounted to the top support beam 410. In an embodiment, one or more sensors 402 can be mounted to the vertical pole 408 and/or the base 406. The sensors 402 can detect exhaust emissions, engine sounds, speed, combinations thereof, or the like, associated with one or more vehicles 414 entering and exiting the geographic area. In an embodiment, the sensors 402 can include one or more image capture devices 121. In an embodiment, the sensor environment 400 can include a wireless antenna 416 for wireless electronic communication of data from the sensors 402 to the remaining components of the system 100.

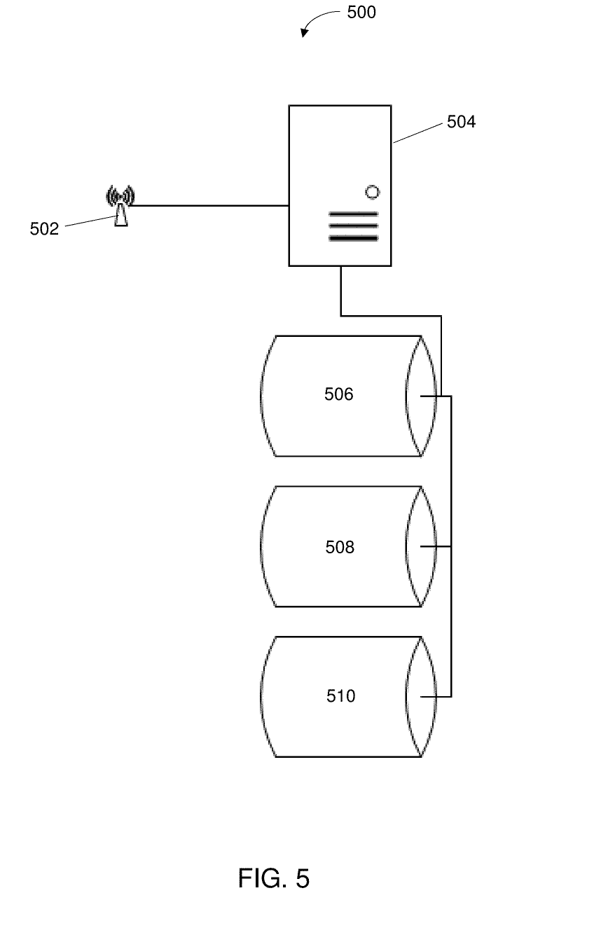

FIG. 5 is a block diagram of an exemplary database system 500 of the system 100 in an embodiment. The database system 500 can include a wireless access point 502 configured to electronically receive and transmit data. For example, the wireless access point 502 can receive data from the exhaust emission sensors 102 and/or the audio sensors 106 for storage within the database system 500. As a further example, the wireless access point 502 can act as a communication interface to transmit data from the database system 500 to the emissions analysis system 108 and/or the engine sound analysis system 132. The database system 500 includes one or more servers 504 configured to transmit the received data for storage in the respective location database 506 (e.g., geographic locations of each of the sensors), exhaust emissions profile database 508 (e.g., a historic database of known exhaust emissions for different vehicle types), and engine sound database 510 (e.g., vehicle engine audio recording database for known engine sounds for different vehicle types).

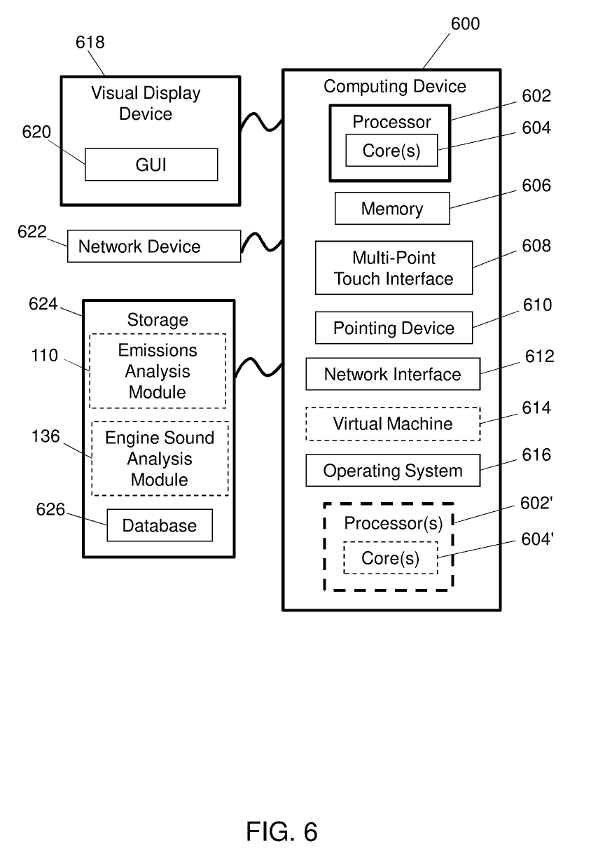

FIG. 6 is a block diagram of a computing device 600 in accordance with exemplary embodiments. The computing device 600 includes one or more non-transitory computer-readable media for storing one or more computer-executable instructions or software for implementing exemplary embodiments. The non-transitory computer-readable media may include, but are not limited to, one or more types of hardware memory, non-transitory tangible media (for example, one or more magnetic storage disks, one or more optical disks, one or more flash drives), and the like. For example, memory 606 included in the computing device 600 may store computer-readable and computer-executable instructions or software for implementing exemplary embodiments of the present disclosure (e.g., instructions for executing the emissions analysis module 110, the engine sound analysis module 136, the identification engine, the correlation engine, combinations thereof, or the like). The computing device 600 also includes configurable and/or programmable processor 602 and associated core 604, and optionally, one or more additional configurable and/or programmable processor(s) 602′ and associated core(s) 604′ (for example, in the case of computer systems having multiple processors/cores), for executing computer-readable and computer-executable instructions or software stored in the memory 606 and other programs for controlling system hardware. Processor 602 and processor(s) 602′ may each be a single core processor or multiple core (604 and 604′) processor.

Virtualization may be employed in the computing device 600 so that infrastructure and resources in the computing device 600 may be shared dynamically. A virtual machine 614 may be provided to handle a process running on multiple processors so that the process appears to be using only one computing resource rather than multiple computing resources. Multiple virtual machines may also be used with one processor.

Memory 606 may include a computer system memory or random access memory, such as DRAM, SRAM, EDO RAM, and the like. Memory 606 may include other types of memory as well, or combinations thereof.

A user may interact with the computing device 600 through a visual display device 618 (e.g., a personal computer, a mobile smart device, or the like), such as a computer monitor, which may display one or more user interfaces 620 (e.g., GUI 140) that may be provided in accordance with exemplary embodiments. The computing device 600 may include other I/O devices for receiving input from a user, for example, a keyboard or any suitable multi-point touch interface 608, a pointing device 610 (e.g., a mouse). The keyboard 608 and the pointing device 610 may be coupled to the visual display device 618. The computing device 600 may include other suitable conventional I/O peripherals.

The computing device 600 may also include one or more storage devices 624, such as a hard-drive, CD-ROM, or other computer readable media, for storing data and computer-readable instructions and/or software that implement exemplary embodiments of the emissions analysis module 110, the engine sound analysis module 136, combinations thereof, or the like, described herein. Exemplary storage device 624 may also store one or more databases 626 for storing any suitable information required to implement exemplary embodiments. For example, exemplary storage device 624 can store one or more databases 626 for storing information, such as data relating to the location database 122, the emissions database 124, the engine sound database 134, the transaction database 128, or the like, and computer-readable instructions and/or software that implement exemplary embodiments described herein. The databases 626 may be updated by manually or automatically at any suitable time to add, delete, and/or update one or more items in the databases.

The computing device 600 can include a network interface 612 configured to interface via one or more network devices 622 with one or more networks, for example, Local Area Network (LAN), Wide Area Network (WAN) or the Internet through a variety of connections including, but not limited to, standard telephone lines, LAN or WAN links (for example, 802.11, T1, T3, 56 kb, X.25), broadband connections (for example, ISDN, Frame Relay, ATM), wireless connections, controller area network (CAN), or some combination of any or all of the above. The network interface 612 may include a built-in network adapter, network interface card, PCMCIA network card, card bus network adapter, wireless network adapter, USB network adapter, modem or any other device suitable for interfacing the computing device 600 to any type of network capable of communication and performing the operations described herein. Moreover, the computing device 600 may be any computer system, such as a workstation, desktop computer, server, laptop, handheld computer, tablet computer (e.g., the iPad™ tablet computer), mobile computing or communication device (e.g., the iPhone™ communication device), or other form of computing or telecommunications device that is capable of communication and that has sufficient processor power and memory capacity to perform the operations described herein.

The computing device 600 may run an operating system 616, such as versions of the Microsoft® Windows® operating systems, the different releases of the Unix and Linux operating systems, versions of the MacOS® for Macintosh computers, embedded operating systems, real-time operating systems, open source operating systems, proprietary operating systems, or other operating systems capable of running on the computing device and performing the operations described herein. In exemplary embodiments, the operating system 616 may be run in native mode or emulated mode. In an exemplary embodiment, the operating system 616 may be run on one or more cloud machine instances.

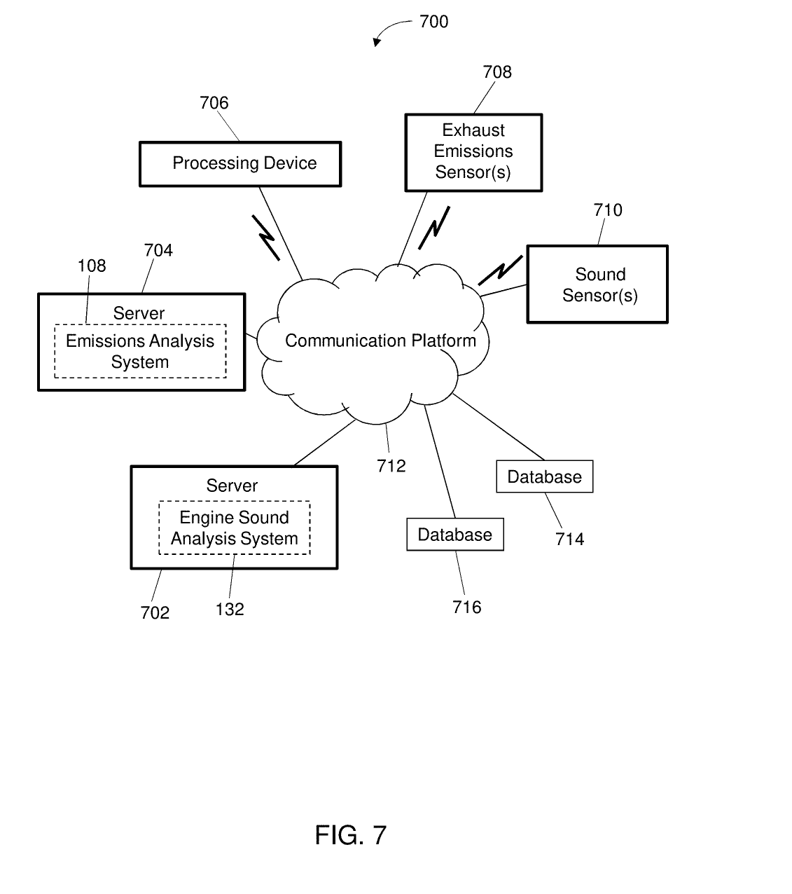

FIG. 7 is a block diagram of an exemplary vehicle identification system environment 700 in accordance with exemplary embodiments of the present disclosure. The environment 700 can include servers 702, 704 operatively coupled to a processing device 706, exhaust emissions sensors 708, and sound sensors 710, via a communication platform 712, which can be any network over which information can be transmitted between devices communicatively coupled to the network. For example, the communication platform 712 can be the Internet, Intranet, virtual private network (VPN), wide area network (WAN), local area network (LAN), and the like. In an embodiment, the communication platform 712 can be part of a cloud environment. The environment 700 can include repositories or databases 714, 716, which can be operatively coupled to the servers 702, 704, as well as to the processing device 706, the exhaust emissions sensors 708, and the sound sensors 710, via the communications platform 712. In exemplary embodiments, the servers 702, 704, processing device 706, exhaust emissions sensors 708, sound sensors 710, and databases 714, 716 can be implemented as computing devices (e.g., computing device 600). Those skilled in the art will recognize that the databases 714, 716 can be incorporated into one or more of the servers 702, 704 such that one or more of the servers 702, 704 can include databases 714, 716. In an embodiment, the database 714 can store the location database 122 and the transaction database 128, and the database 716 can store the emissions database 124 and the engine sound database 134. In an embodiment, a single database 714, 716 can store the location database 122, the emissions database 124, the engine sound database 134, and the transaction database 128.

In an embodiment, embodiments of the servers 702, 704 can be configured to implement one or more portions of the system 100. For example, server 702 can be configured to implement one or more portions of the engine sound analysis system 132. As a further example, server 704 can be configured to implement one or more portions of the emissions analysis system 108.

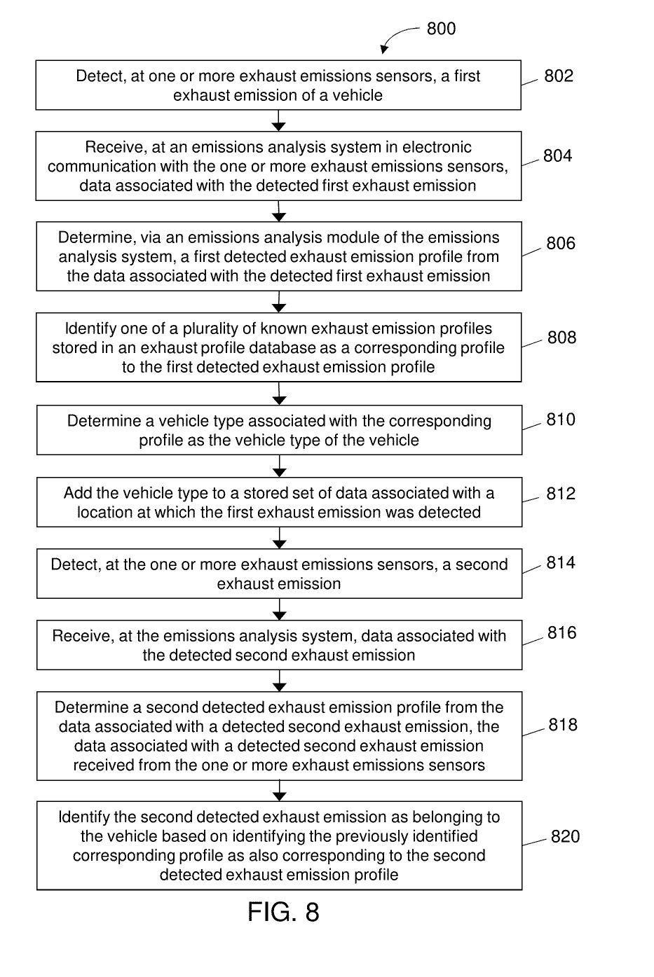

FIG. 8 is a flowchart illustrating an exemplary process 800 as implemented by the vehicle identification system 100 in an embodiment that includes sensors in the form of exhaust emissions sensors. To begin, at step 802, a first exhaust emission of a vehicle can be detected at one or more exhaust emissions sensors. At step 804, data associated with the detected first exhaust emission can be received at an emissions analysis system. At step 806, a first detected exhaust emission profile can be determined via an emissions analysis module from the data associated with the detected first exhaust emission. At step 808, a known exhaust emission profile from a group of known exhaust emission profiles stored in an exhaust profile database can be identified as a corresponding profile to the first detected exhaust emission profile. At step 810, a vehicle type associated with the corresponding profile can be determined as the vehicle type of the vehicle. At step 812, the vehicle type can be added to a stored set of data associated with a location at which the first exhaust emission was detected.

At step 814, a second exhaust emission can be detected at the one or more exhaust emissions sensors. At step 816, data associated with the detected second exhaust emission can be received at the emissions analysis system. At step 818, a second detected exhaust emission profile can be determined from the data associated with the detected second exhaust emission. At step 820, the second detected exhaust emission can be identified as belonging to the vehicle based on identifying the previously identified corresponding profile as also corresponding to the second exhaust emission profile.

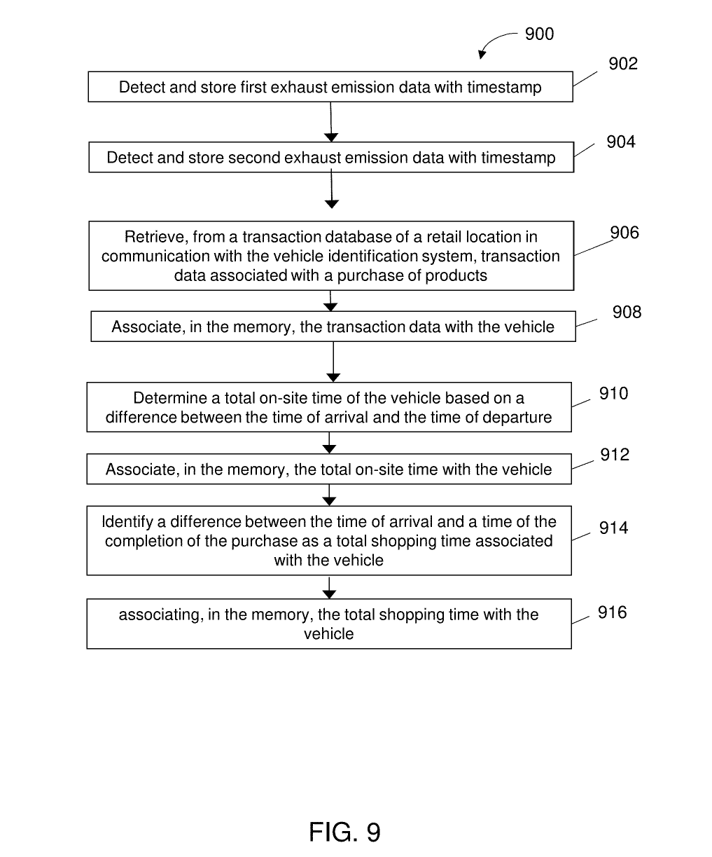

FIG. 9 is a flowchart illustrating an exemplary process 900 as implemented by the vehicle identification system 100 in an embodiment that identifies a shopping time/duration associated with the vehicle. To begin, at step 902, first emission data for a vehicle is detected and stored in a database. The stored data is accompanied with a timestamp indicating the time of detection. At step 904, second emission data for the vehicle is detected and stored in the database. The stored detected second emission data is also accompanied by a timestamp indicating the time of detection. At step 906, transaction data associated with a purchase of products completed subsequent to the detection of the first exhaust emission of the vehicle by the one or more emissions sensors and prior to the detection of the second exhaust emission is retrieved from a transaction database of a retail location in communication with the system 100. At step 908, the transaction data can be associated in the memory with the vehicle. This association may occur in a number of ways. For example, the vehicle may have unique emission characteristics for the time period in question at the retail location that allow the first and second emissions to be associated in the database and the time of the product purchase may enable the purchase to be definitively associated with the vehicle (i.e. the purchase may be the only one that took place in the time window between the first and second detection). Similarly, the association of the purchase to the vehicle identified in the second detection may be probabilistically determined based on the second detection being within a certain time period following the purchase. Alternatively, video analytics in combination with the emission sensors or alone may be used to associate the purchases with the vehicle. As noted above, other techniques such as retrieved customer profile information on file for the vehicle may be matched to purchase information or audio sensors may compare an expected change in engine RPMs from the weight of the purchases to identifiy an associated vehicle.

At step 910, a total on-site time of the vehicle is determined based on a difference between the time of arrival (the time of first detection in one embodiment) and the time of departure of the vehicle (the time of second detection in one embodiment). At step 912, the total on-site time can be associated with the vehicle information in the database. At step 914, a difference between the time of arrival and a time of the completion of the purchase can be identified as a total shopping time associated with the vehicle. At step 916, the total shopping time can be associated with the vehicle information in the database.

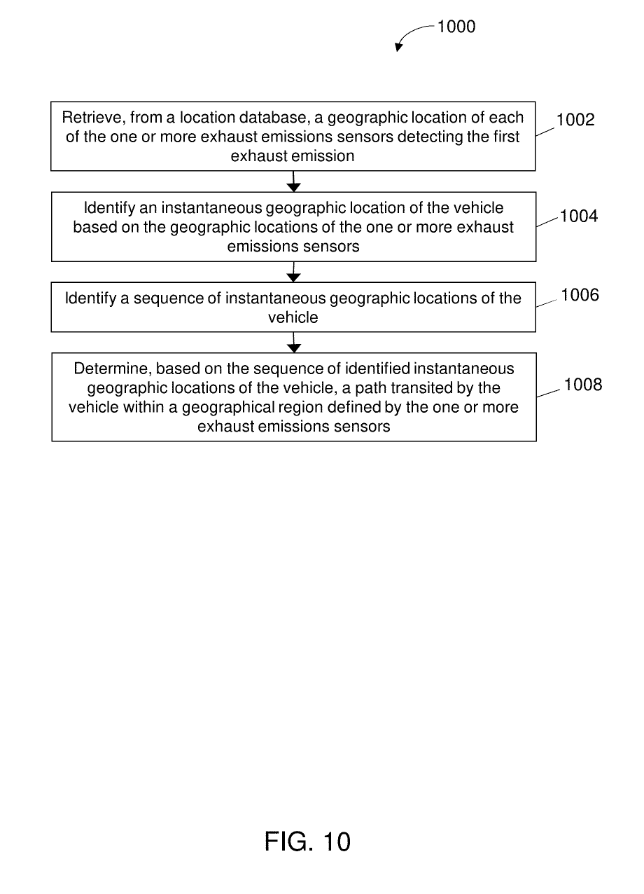

FIG. 10 is a flowchart illustrating an exemplary process 1000 as implemented by the vehicle identification system 100 in an embodiment that identifies a path of the vehicle. The described embodiment includes sensors in the form of exhaust emissions sensors. However, it should be understood that a substantially similar process 1000 can be implemented with sensors in the form of audio or other types of sensors. To begin, at step 1002, a geographic location of each of the one or more exhaust emissions sensors detecting the first exhaust emission can be retrieved from a location database. At step 1004, an instantaneous geographic location of the vehicle can be identified based on the geographic locations of the one or more exhaust emissions sensors. At step 1006, a sequence of instantaneous geographic locations of the vehicle can be identified. At step 1008, based on the sequence of identified instantaneous geographic locations of the vehicle, a path transited by the vehicle within a geographical region or area defined by the one or more exhaust emissions sensors can be determined.

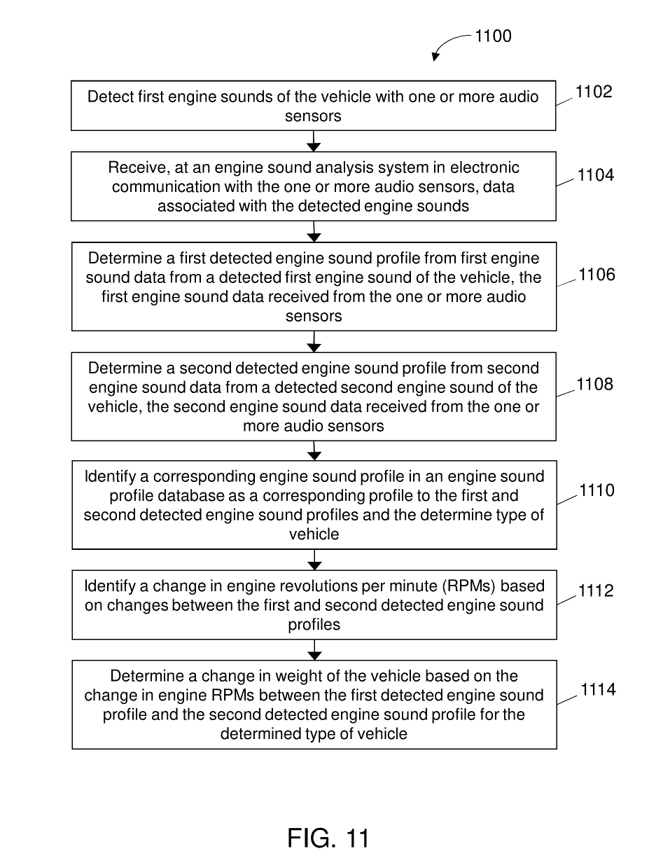

FIG. 11 is a flowchart illustrating an exemplary process 1100 as implemented by the vehicle identification system 100 in an embodiment that includes sensors in the form of audio sensors. To begin, at step 1102, first engine sounds of the vehicle can be detected with the one or more audio sensors. At step 1104, data associated with the detected engine sounds can be received at an engine sound analysis system. At step 1106, a first detected engine sound profile can be determined from the first engine sound data of the detected first engine sound of the vehicle. At step 1108, a second detected engine sound profile can be determined from the second sound data of a detected second engine sound of the vehicle.

At step 1110, a corresponding engine sound profile can be identified in an engine sound profile database as a corresponding profile to the first and second detected engine sound profiles, and the type of vehicle can be determined. At step 1112, a change in engine RPMs can be identified based on changes between the first and second detected engine sound profiles. At step 1114, a change in weight of the vehicle can be determined or estimated based on the change in engine RPMs between the first detected engine sound profile and the second detected sound profile for the determined type of vehicle.

Thus, the exemplary vehicle identification system provides sensors for gathering data regarding customers visiting the retail establishment. In particular, the exhaust emissions sensors identify a vehicle based on exhaust emissions of a vehicle driven by a customer by detecting exhaust emissions of the vehicle at different times including during entry and exit of the vehicle from a geographic area. The exhaust emissions may be used to determine a type and age of vehicle, and to determine the duration of a visit to an establishment of interest. Further, audio sensors may determine a change in engine sounds of the vehicle driven by the customer based on detection of the engine sounds at different times including during entry and exit of the vehicle from the geographic area. Based on such detection, the type of vehicle driven by the customer, the age of the vehicle, and the estimated amount of products purchased by the customer at the retail establishment can be determined. Further still, correlation with transaction data may support the determinations made based on the detected exhaust emissions and/or engine sounds, and improves the overall accuracy of the system.

While exemplary embodiments have been described herein, it is expressly noted that these embodiments should not be construed as limiting, but rather that additions and modifications to what is expressly described herein also are included within the scope of the invention. Moreover, it is to be understood that the features of the various embodiments described herein are not mutually exclusive and can exist in various combinations and permutations, even if such combinations or permutations are not made express herein, without departing from the spirit and scope of the invention.

1. A vehicle identification system, comprising:
one or more exhaust emissions sensors positioned to detect a first exhaust emission of a vehicle; and
an emissions analysis system in electronic communication with and configured to receive data associated with the detected first exhaust emission from the one or more exhaust emissions sensors, the emissions analysis system including:
a location database storing a geographic location of each of the one or more exhaust emissions sensors,
an exhaust profile database storing a plurality of known exhaust emission profiles, each of the plurality of known exhaust emission profiles associated with a known vehicle type,
a processor in a processing device, and
a memory, the memory including instructions for an emissions analysis module that, when executed by the processor, cause the emissions analysis system to:
determine a first detected exhaust emission profile from the data associated with the detected first exhaust emission,
identify one of the plurality of known exhaust emission profiles stored in the exhaust profile database as a corresponding profile to the first detected exhaust emission profile,
determine a vehicle type associated with the corresponding profile as the vehicle type for the vehicle, and
add the vehicle type to a stored set of data associated with a location at which the first exhaust emission was detected.

2. The vehicle identification system of claim 1, wherein the emissions analysis module, when executed by the processor, further causes the emissions analysis system to:
determine a second detected exhaust emission profile from data associated with a detected second exhaust emission, the data associated with a detected second exhaust emission received from the one or more exhaust emissions sensors;
identify the second detected exhaust emission as belonging to the vehicle based on identifying the previously identified corresponding profile as also corresponding to the second detected exhaust emission profile.

3. The vehicle identification system of claim 2, wherein the emissions analysis module further includes instructions that, when executed by the processor, cause the emissions analysis system to:
retrieve, from a transaction database of a retail location in communication with the vehicle identification system, transaction data associated with a purchase of products, the purchase completed subsequent to the detection of the first exhaust emission of the vehicle by the one or more emissions sensors and prior to the detection of the second exhaust emission; and
associate the transaction data with the stored set of data.

4. The vehicle identification system of claim 2, wherein the emissions analysis module further includes instructions that, when executed by the processor, cause the emissions analysis system to:
identify a time of the detection of the first exhaust emission as a time of arrival of the vehicle;
identify a time of the detection of the second exhaust emission as a time of departure of the vehicle;
determine a total on-site time of the vehicle based on a difference between the time of arrival and the time of departure; and
add the total on-site time to the stored set of data.

5. The vehicle identification system of claim 3, wherein the emissions analysis module further includes instructions that, when executed by the processor, cause the emissions analysis system to:
identify a time of the detection of the first exhaust emission as a time of arrival of the vehicle;
identify a difference between the time of arrival and a time of the completion of the purchase as a total shopping time associated with the vehicle; and
add the total shopping time to the stored set of data.

6. The vehicle identification system of claim 3, wherein the emissions analysis module further includes instructions that, when executed by the processor, cause the emissions analysis system to assign the transaction data to a demographic associated with the determined vehicle type.

7. The vehicle identification system of claim 1, wherein the first exhaust emission of the vehicle is detected by at least two of the one or more exhaust emissions sensors, and the emissions analysis module further includes instructions that, when executed by the processor, cause the emissions analysis system to:
retrieve, from the location database, a geographic location of each of the one or more exhaust emissions sensors detecting the first exhaust emission, and
identify an instantaneous geographic location of the vehicle based on the geographic locations of the one or more exhaust emissions sensors.

8. The vehicle identification system of claim 7, wherein the emissions analysis module further includes instructions that, when executed by the processor, cause the emissions analysis system to:
identify a sequence of instantaneous geographic locations of the vehicle;
determine, based on the sequence of identified instantaneous geographic locations of the vehicle, a path transited by the vehicle within a geographical region defined by the one or more exhaust emissions sensors.

9. The vehicle identification system of claim 1, further comprising:
one or more audio sensors positioned to detect engine sounds of the vehicle; and
an engine sound analysis system in electronic communication with and configured to receive engine sound data associated with the detected engine sounds of the vehicle from the one or more audio sensors.

10. The vehicle identification system of claim 9, wherein the engine sounds analysis system includes:
an engine sound profile database storing a plurality of known engine sound profiles, each of the plurality of known engine sound profiles associated with a known vehicle type, and
an engine sound analysis module that, when executed by the processor:
determines a first detected engine sound profile from first engine sound data from a detected first engine sound of the vehicle, the first engine sound data received from the one or more audio sensors,
determines a second detected engine sound profile from second engine sound data from a detected second engine sound of the vehicle, the second engine sound data received from the one or more audio sensors;
identifies a corresponding engine sound profile in the engine sound profile database as a corresponding profile to the first and second detected engine sound profiles and the determined type of vehicle;
identifies a change in engine revolutions per minute (RPMs) based on changes between the first and second detected engine sound profiles; and
determines a change in weight of the vehicle based on the change in engine RPMs between the first detected engine sound profile and the second detected engine sound profile for the determined type of vehicle.

11. A method for vehicle identification, comprising:
detecting, at one or more exhaust emissions sensors, a first exhaust emission of a vehicle;
receiving, at an emissions analysis system in electronic communication with the one or more exhaust emissions sensors, data associated with the detected first exhaust emission;
determining, via an emissions analysis module of the emissions analysis system, a first detected exhaust emission profile from the data associated with the detected first exhaust emission;
identifying one of a plurality of known exhaust emission profiles stored in an exhaust profile database as a corresponding profile to the first detected exhaust emission profile,
determining a vehicle type associated with the corresponding profile as the vehicle type of the vehicle; and
adding the vehicle type to a stored set of data associated with a location at which the first exhaust emission was detected.

12. The method of claim 11, further comprising:
detecting, at the one or more exhaust emissions sensors, a second exhaust emission;
receiving, at the emissions analysis system, data associated with the detected second exhaust emission;
determine a second detected exhaust emission profile from the data associated with a detected second exhaust emission, the data associated with a detected second exhaust emission received from the one or more exhaust emissions sensors; and
identify the second detected exhaust emission as belonging to the vehicle based on identifying the previously identified corresponding profile as also corresponding to the second detected exhaust emission profile.

13. The method of claim 12, further comprising:
retrieving, from a transaction database of a retail location in communication with the vehicle identification system, transaction data associated with a purchase of products completed subsequent to the detection of the first exhaust emission of the vehicle by the one or more emissions sensors and prior to the detection of the second exhaust emission; and
associating, in the memory, the transaction data with the vehicle.

14. The method of claim 12, further comprising:
identifying a time of the detection of the first exhaust emission as a time of arrival of the vehicle;
identifying a time of the detection of the second exhaust emission as a time of departure of the vehicle;
determining a total on-site time of the vehicle based on a difference between the time of arrival and the time of departure; and
associating, in the memory, the total on-site time with the vehicle.

15. The method of claim 12, further comprising:
identifying a time of the detection of the first exhaust emission as a time of arrival of the vehicle;
identifying a difference between the time of arrival and a time of the completion of the purchase as a total shopping time associated with the vehicle; and
associating, in the memory, the total shopping time with the vehicle.

16. The method of claim 11, the first exhaust emission of the vehicle being detected by at least two of the one or more exhaust emissions sensors, the method further comprising:
retrieving, from a location database, a geographic location of each of the one or more exhaust emissions sensors detecting the first exhaust emission; and
identifying an instantaneous geographic location of the vehicle based on the geographic locations of the one or more exhaust emissions sensors.

17. The method of claim 16, further comprising:
identifying a sequence of instantaneous geographic locations of the vehicle;
determining, based on the sequence of identified instantaneous geographic locations of the vehicle, a path transited by the vehicle within a geographical region defined by the one or more exhaust emissions sensors.

18. The method of claim 11, further comprising:
detecting first engine sounds of the vehicle with one or more audio sensors; and
receiving, at an engine sound analysis system in electronic communication with the one or more audio sensors, data associated with the detected engine sounds.

19. The method of claim 18, further comprising:
determining a first detected engine sound profile from first engine sound data from a detected first engine sound of the vehicle, the first engine sound data received from the one or more audio sensors,
determining a second detected engine sound profile from second engine sound data from a detected second engine sound of the vehicle, the second engine sound data received from the one or more audio sensors;
identifying a corresponding engine sound profile in an engine sound profile database as a corresponding profile to the first and second detected engine sound profiles and the determined type of vehicle;
identifying a change in engine revolutions per minute (RPMs) based on changes between the first and second detected engine sound profiles; and
determining a change in weight of the vehicle based on the change in engine RPMs between the first detected engine sound profile and the second detected engine sound profile for the determined type of vehicle.

20. A non-transitory medium storing computer-executable instructions for vehicle identification, the instructions when executed causing at least one processing device to:
detect, at one or more exhaust emissions sensors, a first exhaust emission of a vehicle;
receive, at an emissions analysis system in electronic communication with the one or more exhaust emissions sensors, data associated with the detected first exhaust emission;
determine, via an emissions analysis module of the emissions analysis system, a first detected exhaust emission profile from the data associated with the detected first exhaust emission;
identify one of a plurality of known exhaust emission profiles stored in an exhaust profile database as a corresponding profile to the first detected exhaust emission profile,
determine a vehicle type associated with the corresponding profile as the vehicle type of the vehicle; and
add the vehicle type to a stored set of data associated with a location at which the first exhaust emission was detected.
